(19) United States
(12) Patent Application Publication
(10) Pub. No.: US 2009/0156940 A1
(43) Pub. Date: Jun. 18, 2009

(54) DUAL-LAYER TRANSDUCER FOR RECTILINEAR OR CURVILINEAR THREE-DIMENSIONAL BROADBAND ULTRASOUND

(75) Inventors: Jesse T. Yen, San Gabriel, CA (US); Jong-Seob Jeong, Los Angeles, CA (US); Chi-Hyung Seo, Seattle, WA (US); Samer Awad, Hungtington Park, CA (US)

Correspondence Address: MCDERMOTT WILL & EMERY LLP, 2049 CENTURY PARK EAST, 38th Floor, LOS ANGELES, CA, 90067-3208, US

(73) Assignee: UNIVERSITY OF SOUTHERN CALIFORNIA, Los Angeles, CA (US)

(21) Appl. No.: 12/333,626

(22) Filed: Dec. 12, 2008

Related U.S. Application Data
(60) Provisional application No. 61/013,123, filed on Dec. 12, 2007.

(51) Int. Cl. A61B 8/00; H01L 41/04; H01L 41/22

(52) U.S. Cl. 600/459; 310/336; 29/25.35

(57) ABSTRACT

Dual-layer acoustic transducer array designs, related fabrication methods, and ultrasound imaging techniques are described. The designs include two perpendicular 1-D arrays for clinical 3-D acoustic imaging of targets near the transducer. These targets can include the breast, carotid artery, prostate, and musculoskeletal system among others. The transducer designs reduce the fabrication complexity and the channel count making 3-D rectilinear imaging more realizable. With such designs, an effective N×N 2-D array can be developed using only N transmitters and N receivers. This benefit becomes very significant when N becomes greater than 128, for example. Embodiments/aspects of the present disclosure are directed to fabricating and interconnecting 2-D arrays with a large number of elements (>5,000) for 3-D rectilinear imaging.

### RELATED APPLICATION

This application claims the benefit of U.S. Provisional Application No. 61/013,123 entitled “Dual-Layer Rectilinear or Curvilinear Three-Dimensional Ultrasound with Harmonic Imaging,” filed 12 Dec. 2007, the entire content of which is incorporated herein by reference.

### BACKGROUND

Prior art ultrasound systems and transducer techniques have recently implemented 3-D imaging using 2-D arrays. Commercially available, fully connected 2-D phased arrays for cardiology and obstetrics have emerged in the past several years. Most of these 2-D arrays use piezoceramics such as lead zirconate titanate (PZT) as the active material. Capacitive micro-machined ultrasonic transducers (cMUTs) are also an attractive alternative due to the use of standard silicon integrated circuit technology and the potential for electronic integration. Most of these 2-D arrays have less than 5,000 elements. These probes typically utilize custom integrated circuits in the handle to funnel thousands of elements from a fully connected 2-D phased array to 128 system channels. In contrast, 2-D arrays analogous to 1-D linear arrays with 128 to 256 elements would need 1282 to 2562, or 16,384 to 65,536 elements to scan a rectilinear, box-shaped volume. Such prior art 2-D arrays and techniques have presented problems in interconnecting the elements, particularly as the number of elements is increased.

Previous attempts to develop arrays for 3-D rectilinear imaging mainly focused on suppressing clutter through unique sparse array designs. The designs included a Mills cross, vernier, and staggered patterns . Due to the extreme sparseness of these arrays, however, where the number of elements greatly exceeds the number of system channels, some clutter is unavoidable. The resultant clutter degrades contrast in the acoustic images, resulting in less than optimal image detection because of poor lateral and/or temporal resolution. These results negatively impact the effectiveness of medical ultrasound imaging.

What are desired, therefore, are improved acoustic imaging techniques that improve contrast such that lesions are easily visualized without significantly increasing computational complexity, and/or worsening lateral and/or temporal resolution.

### SUMMARY

Embodiments/aspects of the present disclosure are directed to techniques addressing the limitations noted for the prior art. Such limitations can include difficulties in fabricating and interconnecting 2-D arrays with a large number of elements (>5,000), which have otherwise limited the development of suitable transducers for 3-D rectilinear imaging. Embodiments of the present disclosure address this problem by utilizing a dual-layer transducer array design.

An aspect of the present disclosure is direct to a dual-layer acoustic transducer design include two perpendicular 1-D arrays for clinical 3-D imaging of targets near the transducer. These targets can include the breast, carotid artery, and musculoskeletal system. This transducer design can reduce fabrication complexity and the channel count making 3-D rectilinear imaging more realizable. With this design, an effective N×N 2-D array can be developed using only N transmitters and N receivers. This benefit becomes very significant when N becomes greater than 128, for example. The dual-layer transducer can be rectilinear or curvilinear in exemplary embodiments.

Another aspect of the present disclosure is directed to fabrication methods for dual-layer acoustic transducers. A further aspect of the present disclosure is directed to imaging techniques with such dual-layer acoustic/ultrasonic transducers.

Embodiments of the present disclosure can be implemented in hardware, software, firmware, or any combinations of such, and can be distributed over one or more networks.

Other features and advantages of the present disclosure will be understood upon reading and understanding the detailed description of exemplary embodiments, described herein, in conjunction with reference to the drawings.

### BRIEF DESCRIPTION OF THE DRAWINGS

Aspects of the disclosure may be more fully understood from the following description when read together with the accompanying drawings, which are to be regarded as illustrative in nature, and not as limiting. The drawings are not necessarily to scale, emphasis instead being placed on the principles of the disclosure. In the drawings:

FIG. 1 depicts a 3-D scanning process of a dual-layer transducer array in (A) transmit and (B) in receive, in accordance with exemplary embodiments of the present disclosure;

FIG. 2 depicts simulated on-axis beamplots of a dual-layer transducer with focus (x,y,z)=(0,0,30) mm: (A) depicts a 3-D beamplot; (B) depicts a contour plot with lines at −10, −20, −30, −40, and −50 dB; (C) depicts an azimuthal beamplot; and (D) depicts an elevational beamplot, in accordance with a further embodiment of the present disclosure;

FIG. 3 depicts simulated off-axis beamplots of a dual-layer transducer with focus (x,y,z)=(15,15,30) mm: (A) depicts a 3-D beamplot; (B) depicts a contour plot with lines at −10, −20, −30, −40, and −50 dB; (C) depicts an azimuthal beamplot; and, (D) depicts an elevational beamplot, in accordance with a further embodiment of the present disclosure;

FIG. 4 includes FIGS. 4A-4B, which depict (A) an acoustic stack of a dual-layer transducer, and (B) a schematic of related flexible circuits, in accordance with exemplary embodiments of the present disclosure;

FIG. 5 depicts a photograph of the prototype dual-layer transducer, in accordance with exemplary embodiments of the present disclosure;

FIG. 6 depicts the electrical impedance in air of the dual-layer transducer of FIG. 5 with simulated results indicated by solid lines and experimental results indicated by dashed lines for impedance measurements of the PZT and PVDF layers;

FIG. 7 depicts simulated and experimental time and frequency responses of the pulse-echo signals of the dual-layer transducer of FIG. 5;

FIG. 8 depicts a composite view showing experimental axial wire target images with short-axis in azimuth (A-C) and short axis in elevation (D-F);

FIG. 9 depicts azimuthal and elevational lateral wire target responses, in accordance with an embodiment of the present disclosure;

FIG. 10 depicts a composite view showing experimental cyst images with the cyst short-axis in azimuth (A-C) and the cyst short axis in elevation (D-F), in accordance with an exemplary embodiment of the present disclosure;

FIG. 11 depicts a diagrammatic representation of a method of fabricating a dual layer acoustic transducer array in accordance with an exemplary embodiment of the present disclosure; and

FIG. 12 depicts alternate embodiments of a cylindrical probe including curvilinear ultrasound transducers, in accordance with the present disclosure.

While certain embodiments are depicted in the drawings, one skilled in the art will appreciate that the embodiments depicted are illustrative and that variations of those shown, as well as other embodiments described herein, may be envisioned and practiced within the scope of the present disclosure.

### DETAILED DESCRIPTION

Aspects/embodiments of the present disclosure are generally directed to dual-layer transducer array designs, related fabrication techniques, and related ultrasound imaging techniques. Such dual-layer transducer designs include two perpendicular 1-D arrays in a dual-layer configuration, and can be utilized for clinical 3-D imaging of targets near the transducer. Targets for ultrasound imaging can include, but are not limited to, the breast, carotid artery, prostate, and musculoskeletal system among others. Transducer designs according to the present disclosure can accordingly provide for a reduction in the fabrication complexity and the channel count, making 3-D rectilinear imaging more realizable. With such designs, an effective N×N 2-D array can be developed using only N transmitters and N receivers. This benefit becomes very significant when N becomes greater than 128, for example.

An aspect of the present disclosure is directed to a dual-layer design for 3-D imaging. Such dual-layer designs can utilize one piezoelectric layer for transmit and another separate piezoelectric layer for receive. The receive layer can be closer to the target, and the transmit layer can be configured underneath the receive layer, or vice versa. Each layer can be an elongated 1-D array with the transmit and receive elements oriented perpendicular to each other. The choice of material for each layer can be optimized separately for transmit and for receive. Furthermore, transmit and receive electronics can be isolated. Exemplary embodiments can utilize a dual-layer PZT/P[VDF-TrFE] transducer array for 3-D rectilinear imaging. The transducers can be arranged in flat (rectilinear) or curved (curvilinear) configurations.

A 4×4 cm prototype embodiment of a dual-layer transducer composed of 256 PZT elements and 256 P[VDF-TrFE] elements was developed and tested by the present inventors. Description of the fabrication, test, and initial imaging experiments with this transducer design are described below. 3-D Rectilinear Scanning

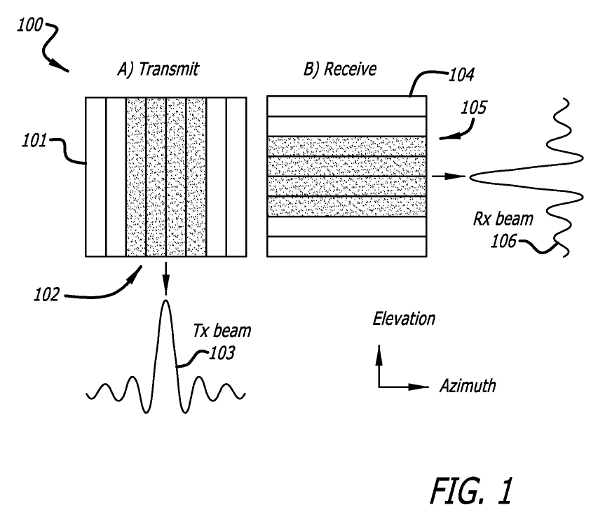

FIG. 1 is a simplified schematic of the rectilinear 3-D scanning process using a dual-layer transducer design 100 with only 8 elements in each layer, in accordance with exemplary embodiments of the present disclosure. Shaded elements indicate active subapertures, such as would be used for scanning. The transmit layer 101 contains a 1-D linear array with elements along the azimuth direction. This transmit array 101 performs beamforming, or focusing, in the azimuth direction using the gray subaperture elements 102 (FIG. 1A), producing focused transmit beam 103.

In receive, a second layer contains 104 a 1-D linear array with elements oriented perpendicular with respect to the transmit array 101. This receive layer 104 is located directly in front of the transmit layer 101. This allows the receive layer 104 to perform beamforming in the elevation direction using the elements shaded in gray 105 (FIG. 1B), producing beamformed receive beam 106.

By moving the locations of transmit and receive subapertures in azimuth and elevation respectively, a rectilinear volume can be scanned for 3-D imaging. Transmit and receive switching between the respective vertical and horizontal electrodes can be accomplished with a simple diode circuit.

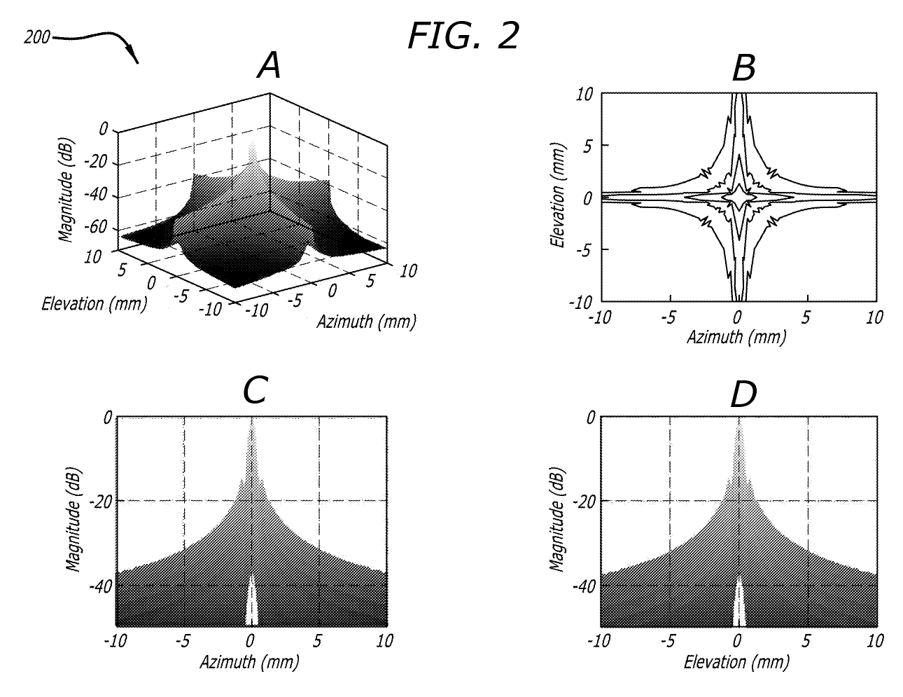

FIG. 2 depicts simulated on-axis beamplots of a dual-layer transducer with focus (x,y,z)=(0,0,30) mm: (A) depicts a 3-D beamplot; (B) depicts a contour plot with lines at −10, −20, −30, −40, and −50 dB; (C) depicts an azimuthal beamplot; and (D) depicts an elevational beamplot.

To evaluate the theoretical imaging performance of embodiments similar to that of FIG. 1, simulated on-axis beamplots were acquired using Field II simulation software, as shown in FIG. 2. The transmit aperture was modeled as a 1-D array with an azimuthal element pitch of one wavelength, or 0.15 mm, and an elevational height of 128 wavelengths, or 38.4 mm. The receive aperture was modeled as having an elevational element pitch of 0.15 mm and an azimuthal length of 38.4 mm. A Gaussian pulse with a center frequency of 5 MHz and 50% -6 dB fractional bandwidth was used. For the beamplot, a 128-element subaperture was used in both transmit and receive and focused on-axis to (x,y,z)=(0,0,30) mm.

As shown in FIG. 2, the resulting −6 dB and −20 dB beamwidths are 0.55 mm and 2.39 mm respectively. The highest clutter levels, around −30 to −40 dB, are seen along the azimuth and elevation axes. The clutter levels drop off dramatically in regions away from the principal azimuth and elevation axes.

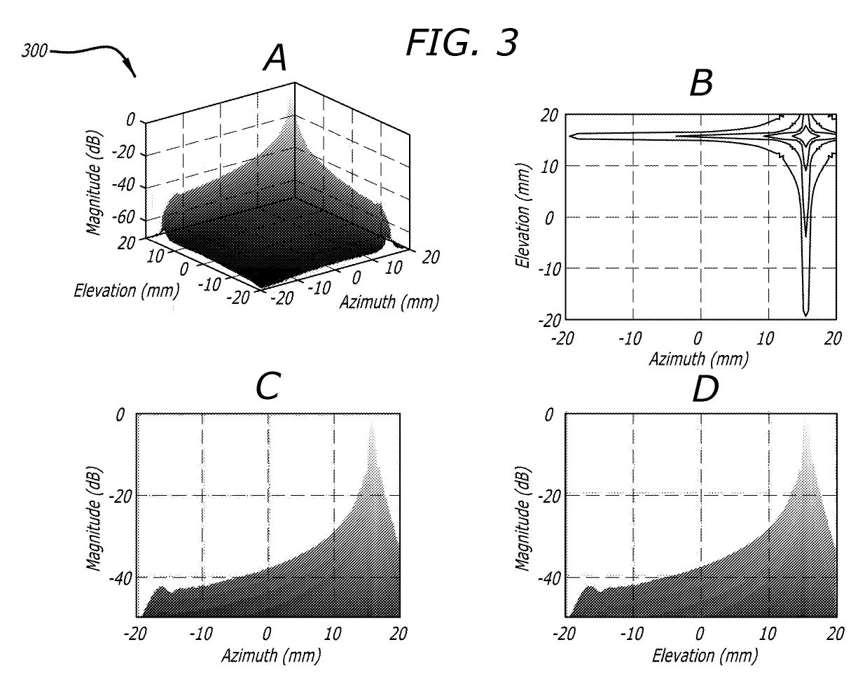

FIG. 3 depicts simulated off-axis beamplots of a dual-layer transducer with focus (x,y,z)=(15,15,30) mm: (A) depicts a 3-D beamplot; (B) depicts a contour plot with lines at −10, −20, −30, −40, and −50 dB; (C) depicts an azimuthal beamplot; and, (D) depicts an elevational beamplot, in accordance with a further embodiment of the present disclosure;

As shown in FIG. 3, for the case of simulated off-axis beamplots when the focus is located at (x,y,z)=(15,15,30) mm, the −6 and −20 dB beamwidths are 0.97 and 4.01 mm respectively. Similar to the on-axis case, the main sources of clutter lie parallel to the azimuthal and elevational axes.

### Dual-Layer Transducer Design and Fabrication

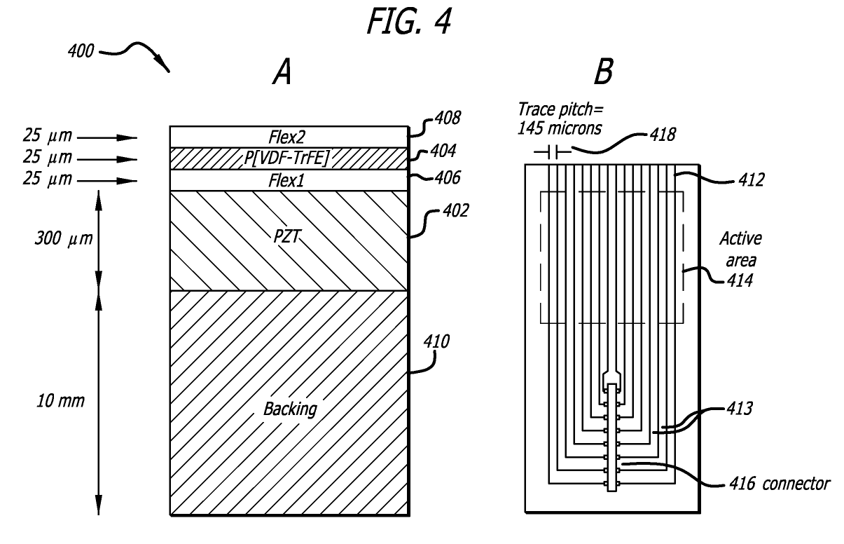

FIG. 4 includes FIGS. 4A-4B, which depict (A) an acoustic stack of a dual-layer transducer 400, and (B) a schematic of related flexible circuits, in accordance with exemplary embodiments of the present disclosure. FIG. 4A shows the acoustic stack of a dual-layer transducer array 400 utilizing PZT and P[VDF-TrFE] materials; other piezoelectric material may be substituted.

As shown in FIG. 4A, the transducer array 400 can include a first layer 402 including a first piezoelectric material, a second layer 404 including a second piezoelectric material, first and second flex circuit layers 406 and 408 each with conductive traces 412, and a backing layer 410. A connector 416 (FIG. 4B) can serve as the interface between the transducer 400 and a printed circuit board (e.g., one suitable for signal processing/ultrasound transmission) with a mating connector.

As shown in FIG. 4B, the two flexible circuits 406 and 408 can be identical with identical patterns of conductive trances 412 for exemplary embodiments. The traces 412 can be arranged in a parallel configuration 413 across an active area 414. Connector 416 can be present, e.g., for coupling to ultrasound generation and processing electronics. The traces 412 can have a desired center-to-center pitch 418.

With continued reference to FIGS. 4A-4B, the first piezoelectric material coupled to the first flex circuit layer forms a flex/piezo layer (e.g., 402 and 406) that when configured with its traces perpendicular to the traces of the other flex/piezo layer (e.g., 404 and 408), formed by the second flex circuit and second piezoelectric material, form an effectively and simply connected 2D ultrasound/acoustic transducer array 400. The simple coupling of the two flex/piezo layers is an advantage over prior art techniques, lending to decreased fabrication costs and ease of construction.

Depending on preference and/or application, the first layer 402 and first flex circuit 406 can be used for transmit or receive, with the same applying to the second layer 404 and second flex circuit 408. Accordingly, for certain applications, the transmit array can be closer to a target/region of interest that the receive layer and vice versa. Moreover, while FIG. 4A indicates that the layer 402 includes PZT and the layer 404 includes P[VDF-TrFE], these are merely representative piezoelectric materials. In some embodiments, the same piezoelectric material may be used in each layer 402 and 404; other piezoelectric materials may be used for each layer in other embodiments.

In an implemented exemplary embodiment, the acoustic stack of the array 400 consisted of a 9.3 MRayl acoustic impedance backing 410, a 300 μm thick PZT-5H layer 402 for transmit, a 25 μm thick prototype flexible circuit 406 (as available from Microconnex, Snoqualmie, Wash.), a 25 μm thick P[VDF-TrFE] copolymer receive layer 404, and another 25 μm thick flexible circuit 408. The layer thickness and the acoustic impedance can be selected as desired, e.g., adjusted based on a desired operational ultrasound frequency or range of frequencies. The flexible circuits 406 and 408 for the embodiment were made of polyimide with 2 μm thick copper traces 414 that were originally designed for a center frequency near 10 MHz, with a center-to-center pitch 418 of 145 μm in an active area 414. Connector 416 (made available Samtec USA, New Albany, Ind.) was used as the interface between the transducer 400 and a printed circuit board with a mating connector.

With continued reference to FIG. 4, an acoustic backing 410 with acoustic impedance of 9.3 MRayl was used, in an exemplary embodiment, to suppress reverberations between transducer layers 406 and 408. This backing 410 was produced using 85% 1 μm tungsten powder (as made available by Atlantic Equipment Engineers, Bergenfield, N.J.) by weight and 15% Epotek 301 epoxy (made available by Epoxy Technology, Billerica, Mass.). The tungsten/epoxy mixture was then centrifuged at 3000 revolutions per minute (rpm) in a Beckman-Coulter Allaegra 6 centrifuge (of Fullerton, Calif.). After lapping to achieve planar surfaces, one side was sputtered with 500 angstroms of chrome and 3000 angstroms of gold to provide a ground plane for all PZT elements.

One skilled in the art will understand that the center-to-center pitch of the conductive traces (e.g., 418 in FIG. 4B) can be designed/selected based on the acoustic frequency/frequencies of interest. Further, the acoustic frequency/frequencies of interest can influence/dictate the selection of the thicknesses (as well as other physical parameters) of layers 402-410 of transducer 400.

For the construction of the implemented embodiment, the PZT layer was formed by first mounting a flexible circuit (Flex1 406 in FIG. 4) to a 5×5 cm glass plate using wax. A 40×40 mm wafer of gold-plated 300 μm thick PZT was then bonded to the flex circuit using nonconductive epoxy. The PZT elements were diced with a 25 μm blade at a pitch of 145 μm. After dicing, the PZT array was bonded to the gold-sputtered side of the backing using Epotek 301, and the glass plate was then removed by melting the wax. Next, a 40×40 mm sheet of copolymer was bonded to another prototype 25 μm thick flex circuit (Flex2 in FIG. 4). This copolymer/flex module was then bonded to the top of Flex1 such that the PZT and copolymer elements were perpendicular to each other. In all bonding steps, the applied pressure was approximately 100 psi.

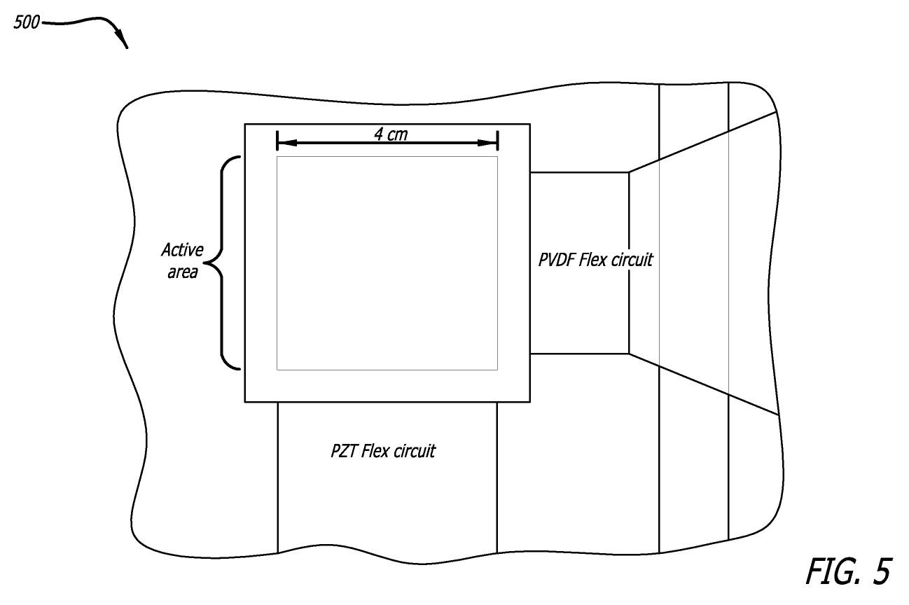

For the embodiments of FIGS. 4 and 5, the copolymer chosen was 25 μm thick, which translates to a half wavelength resonance frequency of 48 MHz. This copolymer thickness was chosen because it has a significantly lower electrical impedance than a thicker copolymer with resonance frequency at 5 MHz. A higher electrical impedance would lower system signal-to-noise ratio (SNR) due to signal loss across the coaxial cable. While a copolymer material thinner than 25 μm could have been used to achieve even lower impedance, this desire was balanced by concerns over handling thinner materials during the transducer fabrication process. Using a 25 μm thickness will give an element impedance roughly equivalent to a PZT 2-D array element. A single copolymer element can be 75 μm wide and 40 mm long, e.g., as in the embodiments shown. These dimensions are defined by the copper trace sizes on the flexible circuit. No dicing was done to the co-polymer layer for the embodiment shown. Overly high crosstalk was not expected since this copolymer has low lateral coupling. The copolymer combines with the two flex circuits (shown as 406 and 408 in FIG. 4) to serve as a simple matching layer for the PZT transmit layer. A photo of the finished prototype transducer 500 is shown in FIG. 5.

After transducer fabrication, electrical impedance measurements were made using an Agilent 4294A (Santa Clara, Calif.) impedance analyzer. Pulse-echo measurements were made in a water tank using a Panametrics 5072PR pulser/receiver (of Waltham, Mass.) with an aluminum plate reflector. To mimic imaging conditions, the excitation pulse was applied to a PZT element and a copolymer element was used as the receiver. Crosstalk measurements of the copolymer and PZT layers were also made using an Agilent 33250A (Santa Clara, Calif.) function generator. A 200 mV_{P-P}, 5 MHz, 20-cycle burst on one element was applied to one element while measuring the voltage on the neighboring element with 1 MΩ coupling on the oscilloscope.

### Data Acquisition

After performing electrical impedance, pulse-echo, and crosstalk experiments, the dual-layer transducer array (transducer 500 of FIG. 5) was interfaced with a Sonix RP ultrasound system (Ultrasonix, Vancouver, Canada) using a custom printed circuit board. This ultrasound system allows the researcher to control imaging parameters such as transmit aperture size, transmit frequency, receive aperture, filtering, and time-gain compensation. In these experiments, one PZT element was connected to one channel of the Sonix system. This channel was used in transmit mode only, and a two-cycle, 5 MHz transmit pulse was used. Sixty-four copolymer elements were each connected to individual system channels configured to operate in receive mode only. With a 40 MHz sampling frequency, data from each receive channel was collected 100 times and averaged to minimize effects of random noise. A different set of 64 receive elements was used until data from all 256 receive elements were collected. This process is repeated until all transmit and receive element combinations were acquired.

### Beamforming, Signal Processing, and Display

The acquired data was then imported into Matlab (Mathworks, Natick, Mass.) for offline 3-D delay-and-sum beamforming, signal processing, and image display. After averaging, dynamic transmit (azimuth) and receive (elevation) focusing was done with 0.5 mm increments with a constant subaperture size of 128 elements, or 18.56 mm.

Beamformed RF data was filtered with a 64-tap bandpass filter with frequency range 3.75-6.25 MHz. A 3-D volume was acquired by selecting the appropriate transmit subapertures in azimuth and receive subapertures in elevation to focus a beam directly ahead.

The rectilinear volume contained 255×255=65,025 image lines with a line spacing of 145 μm in both lateral directions. The dimensions of the acquired volume were 37 (azimuth)×37 (elevation)×45 (axial) mm. After 3-D beamforming, envelope detection was done using the Hilbert transform. Images were then log-compressed and displayed with a dynamic range of 20 to 30 dB. Azimuth and elevation B-scans are displayed along with C-scans which are parallel to the transducer face.

3-D volumes were acquired of custom-made 70×70×70 mm gelatin phantoms containing 5 pairs of nylon wire targets with axial separation of 0.5, 1, 2, 3, and 4 mm. The bottom wire in each pair was laterally shifted by 1 mm with respect to the top wire. This background material of the wire phantom consisted of 400 g DI water, 36.79 g n-propanol, 0.238 g formaldehyde, and 24.02 g gelatin (275 Bloom). These ingredients and quantities are based on recipes given in the literature for evaluating strain imaging techniques. The second phantom imaged had an 8 mm diameter cylindrical anechoic cyst phantom located at a depth of 27 mm from the transducer face. The background of this cyst used the same ingredients as the wire target phantom but with 3.89 g of graphite powder added to provide scattering. For each phantom, two rectilinear volumes were acquired: one with the short axis of the target in the azimuth direction and one with the short axis of the target in the elevation direction.

### Experimental Results

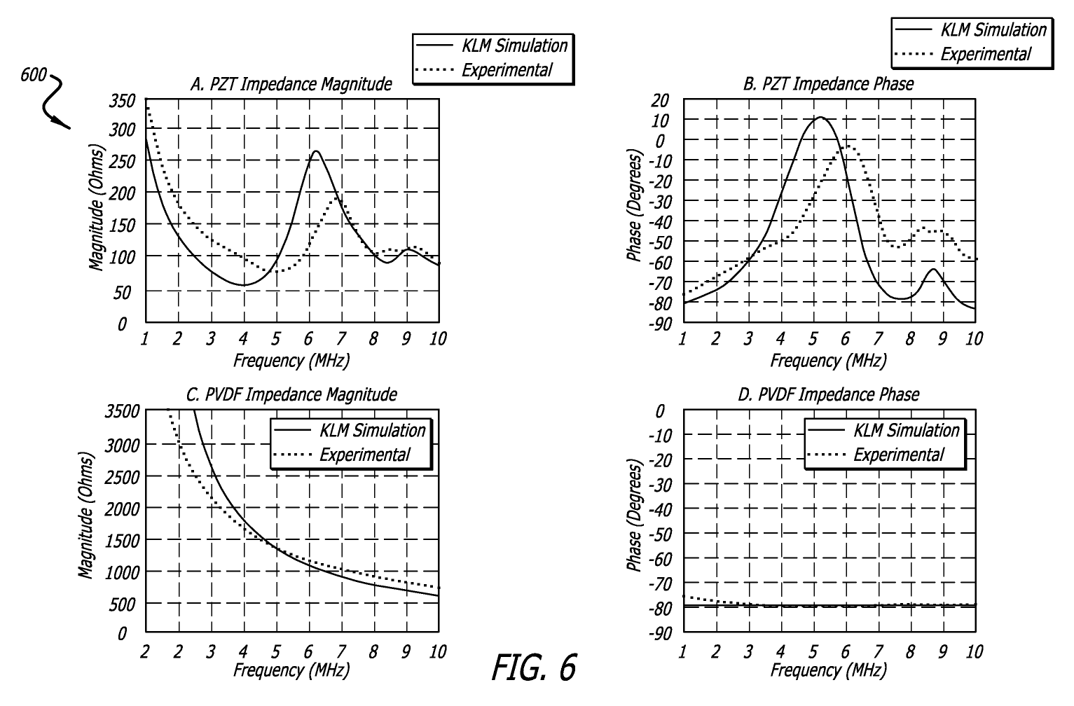

FIG. 6 depicts a combined plot 600 showing the electrical impedance in air of the dual-layer transducer experimentally using an impedance analyzer and by simulation using the 1-D KLM model. For the PZT, the simulated impedance magnitude (shown in A) was 70 Ohms at a series resonance frequency of 4.4 MHz while the experimental impedance curve showed a series resonance of 78 Ohms at 5 MHz. The phase plots (shown in B) peak at 5.5 MHz for the KLM simulation and at 6.04 MHz in the experimental case. The additional resonance in the 8-9 MHz range is most likely due to the flex and copolymer layers. As shown in C, in the simulation, the impedance magnitude of the copolymer was 1.6 kΩ at 5 MHz while the measured impedance magnitude was 1.3 kΩ. As shown in D, no resonance peaks are seen in the impedance magnitudes, and the phase remains near 80° to 85°.

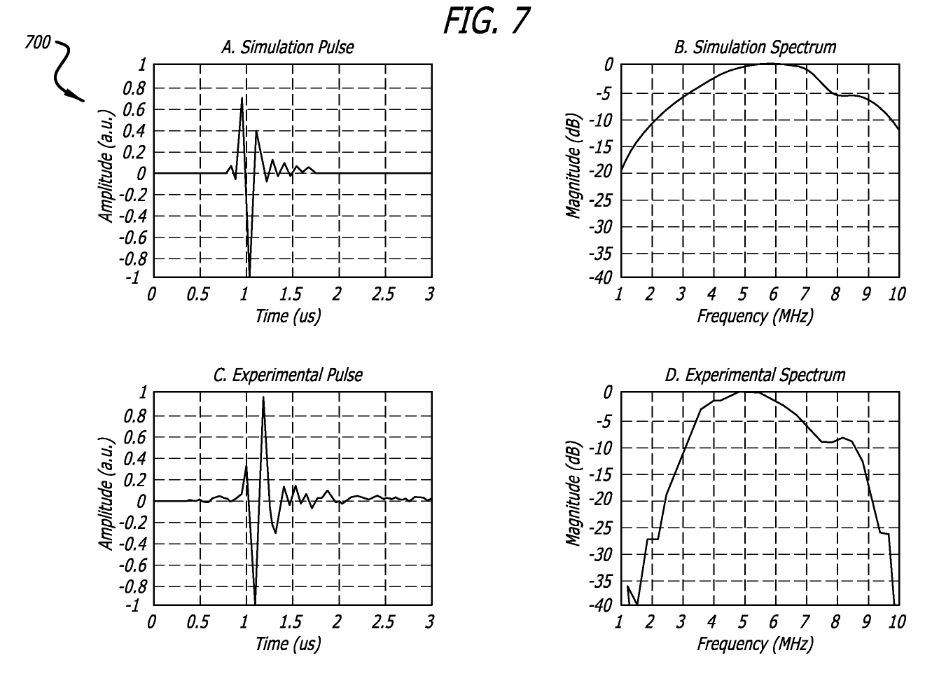

FIG. 7 depicts a combined plot 700 showing simulated and experimental time and frequency responses of the pulse-echo signals of the dual-layer transducer of FIG. 5. In simulation, the center frequency was 5.7 MHz with a −6 dB fractional bandwidth of 90%. Experimentally, the center frequency was 4.8 MHz with a −6 dB fractional bandwidth of 80%. Low amplitude reverberations after the pulse peak are seen in both the simulation and experimental pulses in the time domain. A notch in the 7-8 MHz range is seen in both simulation and experimental spectra. For the PZT layer, the average nearest-neighbor crosstalk at 5 MHz was −30.4±3.1 dB, and the average crosstalk for the copolymer layer was −28.8±3.7 dB. The copolymer layer showed only slightly higher crosstalk than the PZT layer even though no dicing of the copolymer layer was done.

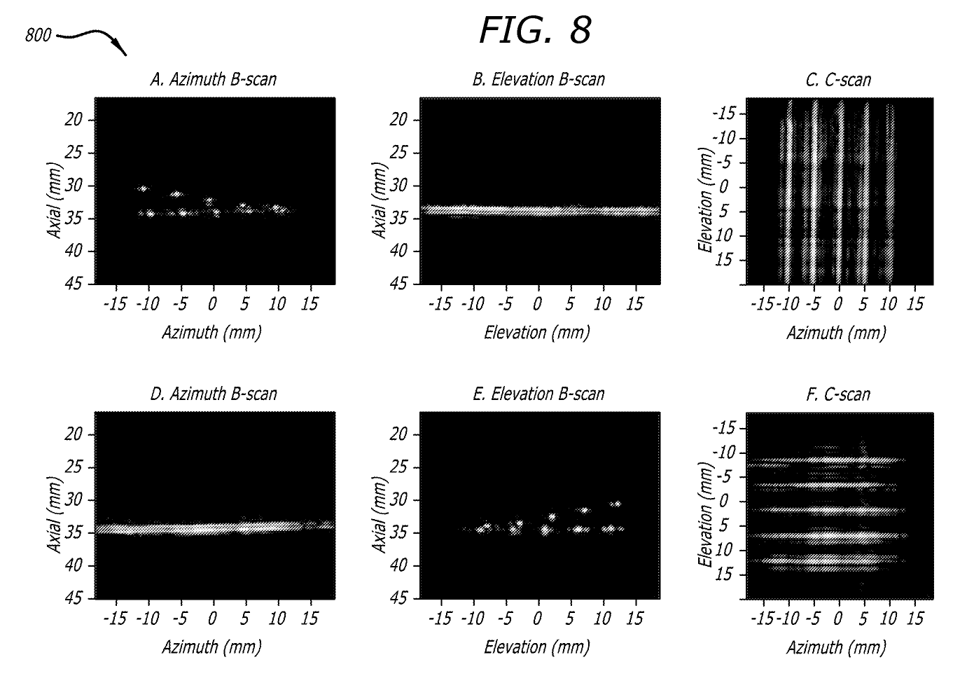

FIG. 8 depicts a composite 800 of FIGS. 8A-8E showing experimental axial wire target images with short-axis in azimuth (A-C) and short axis in elevation (D-F). All images are log-compressed and shown with 20 dB dynamic range.

FIGS. 8A-8C show the azimuth B-scan, elevation B-scan, and C-scan respectively when the short axis of the wires is in the azimuth direction. All images are log-compressed and shown on a 20 dB dynamic range. The elevation B-scan (FIG. 8B) shows the pair of wires with 0.5 mm axial separation. The two wires are discernible. The C-scan, taken at a depth of 35 mm, is parallel to the transducer face. Here, one can also see the presence of sidelobes along side the wires.

FIGS. 8D-F show the axial wire target phantom with the short axis of the wires in the elevation direction. The pair of wires with 0.5 mm axial separation is discernible in the azimuth B-scan while the short-axis view is shown in FIG. 8E. FIG. 8F shows the C-scan where sidelobes are again present.

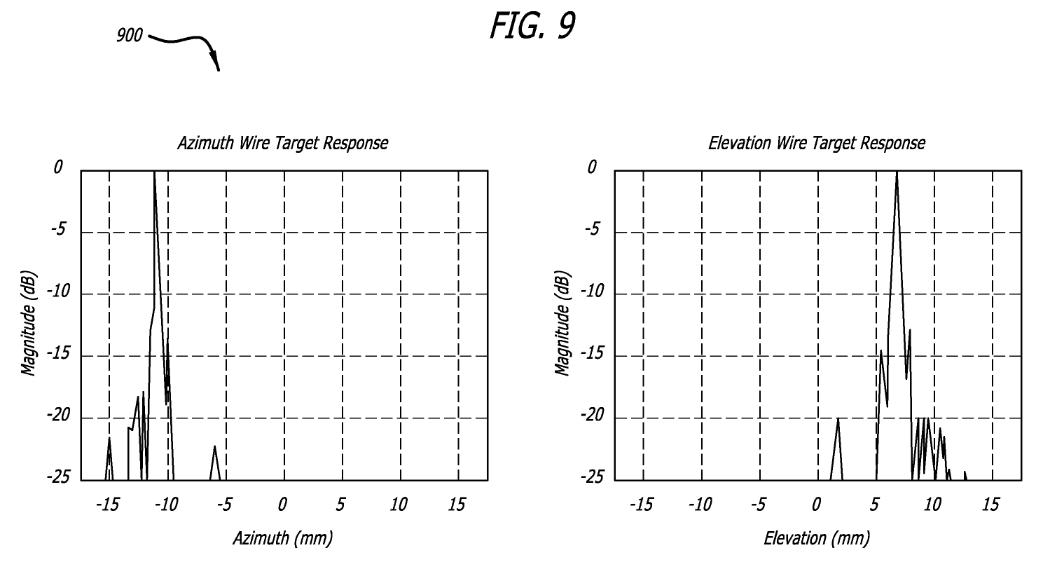

FIG. 9 depicts azimuthal and elevational lateral wire target responses, in accordance with an embodiment of the present disclosure.

FIG. 9 shows a combined plot 900 of the lateral wire target responses in azimuth (FIG. 9A) and elevation (FIG. 9B). In both cases, the wire closest to the transducer was used. The −6 dB beamwidth in azimuth was 0.65 mm and 0.67 mm in elevation compared to a theoretical beamwidth of 0.52 mm in both directions. In both cases, there is a sidelobe above −15 dB and some clutter below −20 dB.

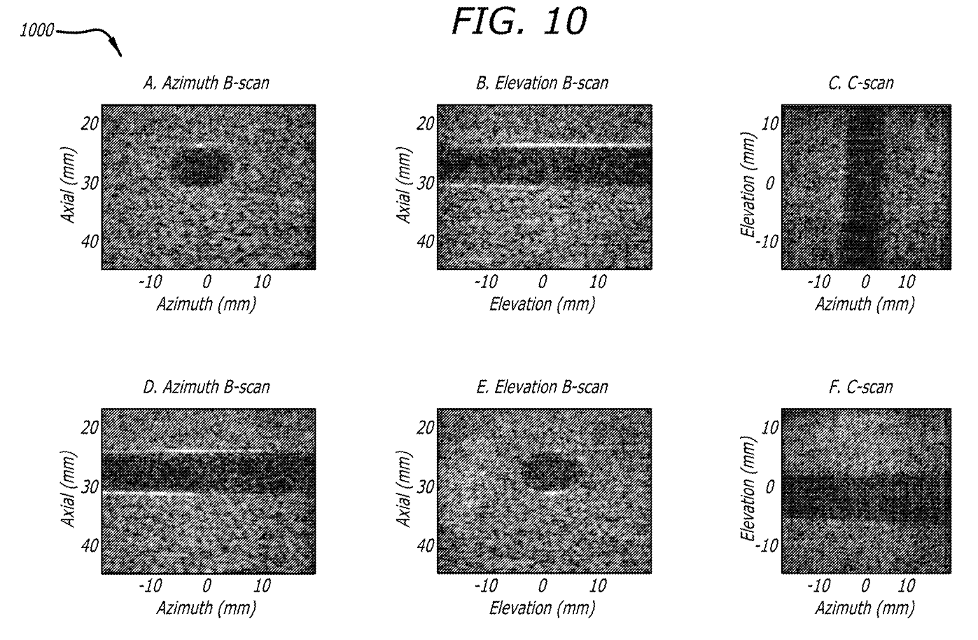

FIG. 10 depicts a composite view 1000 showing experimental cyst images with the cyst short-axis in azimuth (A-C) and the cyst short axis in elevation (D-F), in accordance with an exemplary embodiment of the present disclosure. All images are log-compressed and shown with 30 dB dynamic range. The images of FIG. 10 are phantom images of an 8 mm diameter cyst.

FIG. 10A shows the cyst in cross-section. The cyst is not perfectly circular because of mechanical compression of the phantom to prevent motion during the data acquisition process. In the elevational B-scan and C-scan, the cylindrical cyst appears as a rectangle. FIGS. 10D-F show the cyst with short axis in elevation. Although some clutter is present, the cyst is visible in all images.

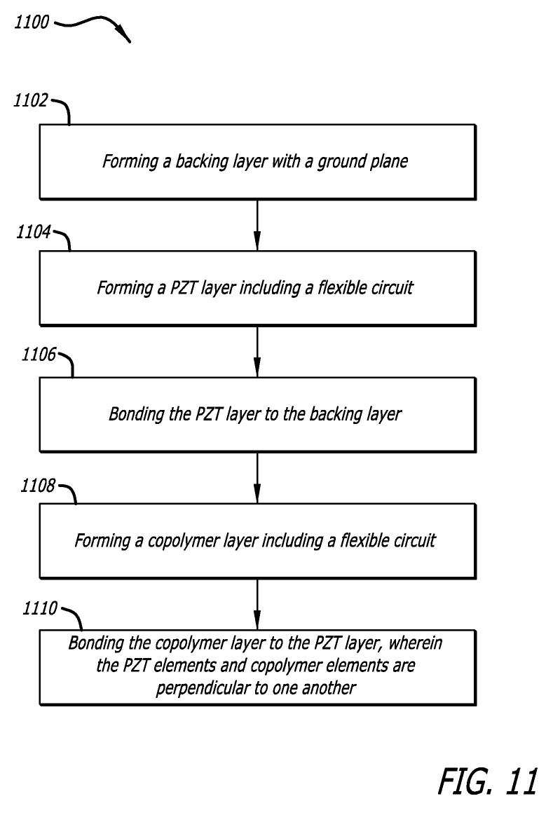

FIG. 11 depicts a diagrammatic representation of a method 1100 of fabricating a dual layer acoustic transducer array in accordance with an exemplary embodiment of the present disclosure. As shown, a backing layer including a ground plane may be formed, as described at 1102. In exemplary embodiments, e.g., as shown and described for FIGS. 4-5, the backing layer can be produced by using 85% 1 μm tungsten powder (as made available by Atlantic Equipment Engineers, Bergenfield, N.J.) by weight and 15% Epotek 301 epoxy (made available by Epoxy Technology, Billerica, Mass.). The tungsten/epoxy mixture can then centrifuged at 3000 revolutions per minute (rpm), e.g., in a Beckman-Coulter Allaegra 6 centrifuge (of Fullerton, Calif.). After lapping to achieve planar surfaces, one side can be sputtered with chrome (e.g., 500 Angstroms thickness) and gold (e.g., 3000 Angstroms thickness) to provide a ground plane for all PZT elements.

Continuing with the description of method 1100, a PZT layer including a flexible circuit (e.g., flexible circuit layer 406 of FIG. 4) can be formed, as described at 1104. In exemplary embodiments, to build the PZT layer, a flexible circuit (e.g., Flex1 406 in FIG. 4) can first be mounted to a 5×5 cm glass plate using wax. A 40×40 mm wafer of gold-plated 300 μm thick PZT can then be bonded to the flex circuit using nonconductive epoxy. The PZT elements can then be diced with a 25 μm blade at a pitch of 145 μm. After dicing, the PZT array can be bonded to the gold-sputtered side of the backing, made at 1102, as described at 1106. The PZT layer can be bonded to the backing layer by suitable epoxy, including Epotek 301. The glass plate can then be removed by melting the wax.

Next, a copolymer later can be fabricated, as described at 1108. In exemplary embodiments, a 40×40 mm sheet of copolymer can be bonded to another 25 μm thick flex circuit (e.g., Flex2 408 in FIG. 4). This copolymer/flex module can then bonded to the top of the first flexible circuit, as described at 1110, such that the PZT and copolymer elements are perpendicular to each other. In all bonding steps, the applied pressure can be approximately 100 psi for exemplary embodiments.

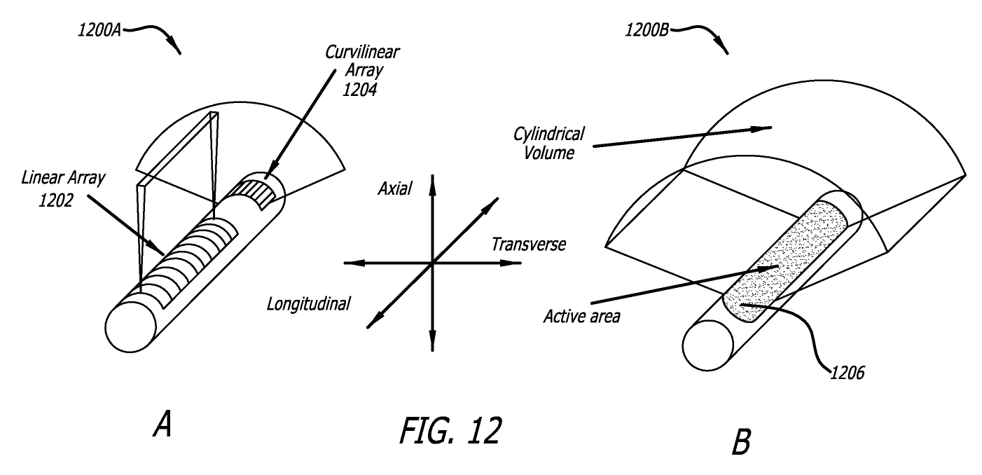

FIG. 12 depicts alternate embodiments 1200A, 1200B of a cylindrical probe including curvilinear ultrasound transducers, in accordance with the present disclosure. Cylindrical probes 1200A and 1200B can be utilized for transrectal ultrasound (“TRUS”) and other medical procedures.

FIG. 12 A depicts a bi-plane probe consisting of two perpendicular 1-D array transducers for transrectal ultrasound (TRUS). Probe 1200A can be used to give two perpendicular B-scans. In one configuration, a flat linear array 1202 can be used to give a rectangular B-scan in the longitudinal direction and a curved linear array 1204 can be used to give a curvilinear B-scan in a plane perpendicular to the long-axis of the probe. In operation, the linear array 1202 would use a subset of elements, or subaperture, to direct a beam directly ahead for each image line. Through multiplexing, this subaperture “walks” from one end of the array to the other. At each step, a new scan line is produced and the scan lines are placed together to form a rectangular image. The curvilinear array operates in a similar manner except that the subaperture moves along in an arc instead of a straight line due to the curved nature of this array. Operating in the 5-10 MHz range, such linear sequential and curvilinear arrays, e.g., might have 128 available elements and a total aperture size of 2-4 cm. A 1-D linear sequential array has transducer elements along one direction only. Consequently, focusing of the ultrasound beam can only be done in this direction.

FIG. 12B depicts a probe 1200B utilizing a curvilinear dual-layer transducer 1206 (e.g., a curvilinear configuration of transducer 400 of FIG. 4), in accordance with the present disclosure. Used for 3-D transrectal ultrasound, transducer 1206 could acquire a cylindrical volume large enough to capture the entire prostate and/or surrounding tissues (FIG. 1B). Once inserted into the rectum or other body portion, no further manipulation would be required. Visualization of the prostate as well as guidance of minimally invasive procedures can conseuqently be improved.

Accordingly, embodiments of the present disclosure can offer advantages over prior art techniques, including providing reduced fabrication complexity and a decreased number of channels compared to a fully sampled 2-D array of comparable size.

While certain embodiments have been described herein, it will be understood by one skilled in the art that the methods, systems, and apparatus of the present disclosure may be embodied in other specific forms without departing from the spirit thereof. For example, while copolymer layers have been described herein in the context of P[VDF-TrFE], other electroactive polymers such P(VDF-CTFE), P(VDF-TrFE)/P(VDF-CTFE) copolymer blends, and the like may be used.

For additional example, further embodiments can be designed to operate as dual-layer transducers at frequencies higher than 5 MHz (8-14 MHz). Frequencies greater than 5 MHz are more commonly used clinically for imaging targets near the transducer such as the breast, carotid, and musculoskeletal system. Higher frequency dual-layer transducers can include use of a thinner piezoelectric material layer (e.g., PZT), but the same copolymer material and thickness could be used. At higher frequencies, the copolymer material may exhibit lower electrical impedance making the material a better match to system electronics. To improve SNR, low-noise pre-amplifiers could be placed near the elements to drive the coaxial cable. Such designs can be utilized, e.g., for 3-D transrectal imaging of the prostate. In such applications, a cylindrical backing can be made fabricated, and the two perpendicular piezoelectric layers can be curved around this cylindrical backing. The dicing direction of the transmit PZT layer can be parallel to the long axis of the probe. Since copolymer of this thickness is very flexible, it can easily be molded around the cylindrical backing. Other embodiments may also be realized within the scope of the present disclosure.

Accordingly, the embodiments described herein, and as claimed in the attached claims, are to be considered in all respects as illustrative of the present disclosure and not restrictive.

What is claimed is:

1. A dual layer acoustic transducer array comprising:
a first array layer including a first piezoelectric material and configured and arranged to transmit an acoustic beam;
a receive array layer including a second piezoelectric material and configured and arranged to receive a reflection of the acoustic beam;
first and second flexible circuit layers, wherein the first flexible circuit layer and second flexible circuit layer each comprise a plurality of conductive traces configured and arranged substantially parallel to one another within the respective flexible circuit layer, and wherein the plurality of conductive trances of the first flexible circuit layer are substantially perpendicular to the plurality of conductive traces of the second flexible circuit layer; and
a backing layer made of a material with a desired acoustic impedance.

2. The transducer array of claim 1, wherein the first piezoelectric material comprises PZT-5H.

3. The transducer array of claim 1, wherein the second piezoelectric material comprises P[VDF-TrFE] copolymer.

4. The transducer array of claim 1, wherein the first layer is a transmit layer comprising a plurality of transmit elements.

5. The transducer array of claim 1, wherein the first layer is a receive layer.

6. The transducer array of claim 1, wherein the second layer is a transmit layer comprising a plurality of transmit elements.

7. The transducer array of claim 1, wherein the second layer is a receive layer.

8. The transducer array of claim 1, wherein the backing layer has an acoustic impedance of about 9.3 MRayl.

9. The transducer array of claim 1, wherein the backing layer comprises about 85% tungsten powder by weight and 15% epoxy by weight.

10. The transducer array of array of claim 9, wherein the tungsten powder has mean particle diameter of about 1 μm.

11. The transducer array of claim 1, wherein the first and second flexible circuit layers are substantially identical.

12. The transducer array of claim 1, wherein the conductive traces have a center-to-center pitch configured and arranged to accommodate a desired frequency of acoustic energy.

13. The transducer array of claim 1, wherein the first and second flexible circuit layers comprise polyimide.

14. The transducer array of claim 1, wherein the first and second flexible circuit layers are about 25 μm thick.

15. The transducer array of claim 1, wherein the first and second flexible circuit layers comprise 2 μm thick copper traces configured and arranged for a center frequency of about 10 MHz, with a center-to-center pitch of 145 μm in an active area.

16. The transducer array of claim 1, wherein the backing layer comprises gold.

17. The transducer array of claim 1, wherein the backing layer comprises tungsten.

18. The transducer array of claim 4, wherein the transmit layer comprises a plurality of PZT elements separated from one another.

19. The transducer array of claim 18, wherein the center-to-center spacing of the plurality of PZT elements is configured and arranged to accommodate a desired frequency of acoustic energy.

20. The transducer array of claim 19, wherein the frequency is about 10 MHz.

21. The transducer array of claim 1, wherein the transducer array is rectilinear.

22. The transducer array of claim 1, wherein the transducer is curvilinear.

23. A method of fabricating a dual-layer transducer array for acoustic imaging, the method comprising:
forming a backing layer having a desired acoustic impedance and a ground plane;
forming a transmit array having a first piezoelectric material;
providing a first flexible circuit having a plurality of conductive traces configured and arranged substantially parallel to one another;
attaching the transmit array to the first flexible circuit and forming a flexible transmit layer;
attaching a second flexible circuit to a receive layer having a second piezoelectric material and forming a flexible receive layer;
attaching the flexible receive layer to the flexible transmit layer, wherein the plurality of conductive traces of the flexible receive layer are substantially perpendicular to the plurality of conductive trances of the flexible transmit layer and forming a dual-layer 2-D array module; and
attaching the dual-layer 2-D array module to the backing layer.

24. The method of claim 23, wherein the second piezoelectric material comprises a copolymer.

25. The method of claim 23, wherein the first piezoelectric material comprises PZT.

26. The method of claim 24, wherein the second piezoelectric material comprises P[VDF-TrFE] copolymer.

27. The method of claim 23, wherein forming a transmit array with a first piezoelectric material comprises dicing a piezoelectric wafer into a plurality of parallel elements having a center-to-center pitch configured and arranged to accommodate a desired frequency of acoustic energy.

28. The method of claim 23, wherein attaching the transmit array to the first flexible circuit comprises using epoxy.

29. The method of claim 23, wherein attaching the second flexible circuit to the receive layer comprises using epoxy.

30. The method of claim 23, wherein attaching the flexible receive layer to the flexible transmit layer comprises using epoxy.

31. The method of claim 23, wherein attaching the dual-layer 2-D array module to the backing layer comprises using epoxy.

32. The method of claim 23, wherein the transducer array is rectilinear.

33. The method of claim 23, wherein the transducer is curvilinear.

34. A method of ultrasound imaging comprising:
transmitting acoustic energy of a desired frequency from a flexible transmit layer having a plurality of transmit elements;
receiving reflected acoustic energy with a flexible receive layer having a plurality of receive elements; and
performing signal processing and acquiring a 3-D volume representing an acoustic image;
wherein the flexible receive layer includes a transmit array with a first piezoelectric material and a first flexible circuit having a plurality of conductive traces configured and arranged substantially parallel to one another, wherein the flexible transmit layer includes a copolymer layer with a second piezoelectric material and a second flexible circuit having a plurality of conductive traces configured and arranged substantially parallel to one another, and wherein the flexible receive layer is connected to the flexible transmit layer such that the plurality of conductive traces of the flexible receive layer are substantially perpendicular to the plurality of conductive trances of the flexible transmit layer.

35. The method of claim 34, wherein the first piezoelectric material comprises PZT.

36. The method of claim 34, wherein the second piezoelectric material comprises P[VDF-TrFE] copolymer.

37. The method of claim 34, wherein acquiring a 3-D volume comprises selecting desired transmit subapertures in azimuth and desired receive subapertures in elevation.

38. The method of claim 34, further comprising performing envelope detection.

39. The method of claim 38, wherein performing envelope detection comprises using a Hilbert transform.

40. The method of claim 34, further comprising displaying an image.

42. The method of claim 34, wherein a backing layer having a desired acoustic impedance is attached to the flexible transmit layer or flexible receive layer, forming an acoustic stack.

43. The method of claim 34, wherein the conductive traces of the first and second flexible circuits have a center-to-center pitch configured and arranged to accommodate a desired frequency of acoustic energy.

44. The method of claim 34, wherein the desired frequency is about 5 MHz.

45. The method of claim 42, wherein the acoustic stack is rectilinear.

46. The method of claim 42, wherein the acoustic stack is curvilinear.
